(19) United States
(12) Patent Application Publication
(10) Pub. No.: US 2017/0188640 A1
(43) Pub. Date: Jul. 6, 2017

(54) SIMPLIFIED CARDIOPULMONARY LIGHTNING PROTECTION GARMENT

(71) Applicant: Dan SCHLAGER, (US)

(72) Inventors: Dan SCHLAGER, Tucson, AZ (US); Delmas R BUCKLEY, JR., Livermore, CA (US)

(73) Assignee: Dan SCHLAGER, Tucson, AZ (US)

(21) Appl. No.: 15/466,341

(22) Filed: Mar. 22, 2017

Related U.S. Application Data
(63) Continuation-in-part of application No. 14/591,416, filed on Jan. 7, 2015, now abandoned.
Application No. 14/591,416 is a continuation-in-part of application No. 13/468,069, filed on May 10, 2012, now Pat. No. 9,301,558.
Application No. 13/468,069 is a continuation-in-part of application No. 15/055,927, filed on Feb. 29, 2016, now Pat. No. 9,648,913.
Application No. 15/055,927 is a continuation of application No. 13/468,069, filed on May 10, 2012, now Pat. No. 9,301,558.
(60) Provisional application No. 61/925,077, filed on Jan. 8, 2014.

(51) Int. Cl. A41D 13/008; A42B 3/06; A61F 9/06; A63B 71/10; A45F 3/04; A42B 1/04; A41D 1/04; A41D 3/00; A41D 1/06; A41D 31/00; F41H 1/04

(52) U.S. Cl. CPC A41D 13/008; A41D 31/0072; A42B 3/063; A61F 9/06; A63B 71/10; F41H 1/04; A42B 1/046; A41D 1/04; A41D 3/00; A41D 1/06; A41D 31/0027; A45F 3/04; A41D 2200/20

(57) ABSTRACT

A cardiopulmonary protective garment for providing limited protection from lightning is fabricated of a waterproof/breathable fabric, such as a Gore-Tex® laminate or equivalent. The garment keeps a wearer's body dry and supports a lightning flashover event when moist or wet on the outside, protecting the cardiopulmonary system. Strips of an electrically conductive fabric, such as Shieldex® or equivalent, can be attached in various arrangements on a posterior outer surface of the garment for igniting a rapid flashover. The conductive strips also provide attractive design details. In some embodiments, an inner heat shielding and flame resistant layer, made of a fabric such as NOMEX® or equivalent, can increase protection against burning caused by lightning and a flashover. Other embodiments may include a grounding strap electrically connected to the conductive strips for carrying charge to a local ground plane, such as the Earth; a cape-like drape of water-absorbing fabric having an electrically conductive element attached to a posterior side in general alignment with the wearer's spine; and a water-proof backpack having electrically conductive elements positioned to promote rapid flashover.

### RELATED APPLICATION AND CLAIM OF PRIORITY

This patent application is a continuation-in-part of co-pending U.S. patent application Ser. No. 15/055,927, filed Feb. 29, 2016, entitled “Cardiopulmonary Lightning Protection Garment,” which claims priority as a continuation of U.S. patent application Ser. No. 13/468,069, filed May 10, 2012, entitled “Cardiopulmonary Lightning Protection Garment.” This patent application further claims priority as a continuation-in-part of co-pending U.S. patent application Ser. No. 14/591,416, filed Jan. 7, 2015, entitled “Simplified Cardiopulmonary Lightning Protection Garment,” which claims priority as a continuation-in-part of U.S. patent application Ser. No. 13/468,069, filed May 10, 2012, entitled “Cardiopulmonary Lightning Protection Garment,” and which claims priority from the filing date of U.S. Provisional Patent Application, Ser. No. 61/925,077, filed 8 Jan. 2014, entitled “Simplified Cardiopulmonary Lightning Protection Garment,” the disclosures of which are incorporated herein in their entireties.

### FIELD OF THE INVENTION

The invention relates to protective clothing, and more particularly to garments providing protection against lightning-caused cardiopulmonary arrest.

### BACKGROUND ART

Though a rare cause of death, lightning is reported to be responsible for more fatalities each year in this country than any other type of natural disaster. Lightning injuries differ significantly from other high voltage electrical injuries because of the high current flow, but extremely short duration of the lightning strike.

Lightning can affect all organ systems, especially the cardiovascular system. The primary cause of death following a lighting strike is cardiopulmonary arrest. The current in a lightning bolt is as high as 30,000 Amperes at 1,000,000 or more Volts. The short duration of about 1-100 milliseconds limits, but does not prevent serious injury. There are several types of outdoors lightning injury. The most severe is a direct strike, either on the victim or on some object the victim is holding such as a golf club, tripod or umbrella. A “side flash” occurs when lightning hits a nearby object and jumps to the victim. Ground current injuries occur when lightning strikes the ground nearby and spreads to a victim.

Avoidance and prevention are the best means of lightning safety. The risk of a lightning related injury can be minimized with some simple safety measures but not eliminated completely. Just as remaining in a metal vehicle during lightning activity can provide protection, a protective garment that includes an electrically conductive shield can benefit someone who finds himself exposed to a potential lightning strike. What is needed is a protective garment that reduces deaths related to cardiopulmonary arrest following a lightning strike.

Applicant has found no patent nor non-patent literature expressly describing such a garment, however, U.S. Pat. No. 7,712,149 issued to Baldwin in 2010 for a “Protective Article of Outer Clothing” discloses a garment intended for protection against attack by Taser (an electrical stun gun), and suggests (at col. 4, lines 8-12) that such garment might be useful as protection against a lightning strike. The Baldwin garment provides an electrically conductive shield, but lacks any other feature that will benefit the victim of a lightning strike.

Several U.S. patents, in addition to that by Baldwin, disclose electrically conductive textiles of varying types, potentially useful for making electrically protective garments, e.g., U.S. Pat. Nos. 7,947,773, 7,832,983, 7,817,401, 7,284,280, 6,272,781, and

U.S. Pat. No. 5,906,004. Medical information related to lightning-caused injury and death has been reported in two publications, limited portions of which are paraphrased above: (1) a publication of the Center for Disease Control, “Lightning-Associated Deaths—United States, 1980-1995”, MMWR 47(19), at pages 391-394, May 22, 1998; and (2) a paper titled “Deaths Caused by Lightning”, by Lifschultz et al., Journal of Forensic Sciences 38(2), at pages 353-358, March 1993.

An interesting medical study published in 1986 [“Lightning injury caused by discharges accompanying flashovers-a clinical and experimental study of death and survival”, Ohashi M., et al., in Burns Incl Therm lnj 1986 October; 12(7): 496-501, Abstract] reported that “[d]uring the 17 years preceding March 1985, 140 patients sustained lightning injuries caused by 44 thunderbolts. Fifty patients showed evidence of current flow through their bodies. These 50 victims were classified into two groups, the first consisting of 9 victims who showed rupture of their clothes or lineal superficial dermal burns along their whole bodies from head to feet, indicating the occurrence of surface flashovers. The remaining 41 patients showed no evidence of this flash effect. It is noteworthy that in the first group 5 of the 9 survived, whereas in the second group only 6 among 41 survived. The result indicates that when a flashover occurs along the whole body, the probability of survival is higher than 50 percent. The conditions which determine death or survival were investigated experimentally, imposing artificial lightning voltage impulses on rats and developing flashovers on them. The rats survived when the voltage drop caused by flashover occurred immediately after the peak point, and the current waveform exhibited a sharp peak. In contrast, the rats were killed when the voltage drop caused by flashover was delayed by more than 20 microseconds, and the current waveform showed a blunt cone shape. It has been concluded that a fast flashover appreciably diminishes the energy dissipation within the body and consequently results in survival.

No garment that is both practical and wearable can prevent most serious injuries resulting from a lightning strike. A victim will be severely injured. What is needed is a protective garment that can reduce the number of deaths resulting each year from cardiopulmonary arrest following a lightning strike.

It is an object of the present invention to provide a garment that can reduce the number of lightning caused deaths by effectively protecting the user's cardiopulmonary system.

### SUMMARY OF THE INVENTION

The above object is achieved by a cardiopulmonary lightning protection garment including an electrically conductive shield covering at least an upper portion of the body and having a region of limited conductivity for directing electrical charge away from the heart while promoting fast flashover. The garment includes a grounding member providing a movable connection between the garment's conductive shield and a local ground plane, such as the Earth.

The principle of operation of the invention is to shield the body while promoting fast flashover to reduce electrical potential, conduct the electricity across the garment body, away from the heart, then down the grounding member to the ground plane- or in a reverse direction for a lightning strike that jumps from Earth to sky.

Various configurations of the basic garment are contemplated, including a hooded jacket, hooded raincoat, padded vest, rain poncho, and the like. In various embodiments, the grounding member is a strap-like tail attached to the electrically conductive body shield at an upper end, and falling to the ground at a lower end. A ball or sliding weight is attached to the lower end of the tail to maintain a movable contact between the conductive tail and the ground, and both ball and tail can be stuffed into a garment pocket for carrying when not in use.

### BRIEF DESCRIPTION OF THE DRAWING

FIG. 1 is a pictorial side view illustrating a cardiopulmonary lightning protection garment providing a fast flashover promoting, electrically conductive body shield (not illustrated) and a grounding strap with weighted end.

FIG. 2 is a partial side view that illustrates an electrically conductive layer enclosed between cloth layers for use in manufacture of the protective garment of FIG. 1.

FIG. 3 is a partial front view illustrating a region of the conductive body shield having a reduced conductivity in the vicinity of the heart.

FIG. 4 is a partial side view illustrating an electrical connection between a conductive body shield and a grounding strap made of a flexible conductor, and illustrating a weighted end.

FIG. 5 is a partial plan view that illustrates an alternative configuration for a grounding strap made of a cloth-covered extension of the conductive body shield, and illustrating a weighted end.

FIGS. 6A-6D illustrate alternative arrangements of a reduced conductivity region near the heart.

FIGS. 7A-7C illustrate an alternative construction for the lower end of the grounding strap, that is here shown attached to the heel of the shoe rather than having a weighted lower end.

FIGS. 8A-8D illustrate details of a cardiopulmonary lightning protection garment in which a waterproof/breathable outer layer provides support for a flashover event when wet, and electrically conductive fabric strips on the outer garment posterior surface serve to promote a rapid flashover.

FIG. 9 illustrates an embodiment of a simple protection garment worn over separate raingear and operating on the same principle as the garment illustrated in FIGS. 8A-8D.

FIG. 10 illustrates an alternative embodiment of the simple protection garment of FIG. 9 configured as a vest.

FIGS. 11A-11B illustrate an alternative embodiment of the simple protection garment of FIG. 9 configured as a shirt, and including a user's belt providing a detachable grounding strap.

FIG. 12 is a rear-side view that illustrates an alternative embodiment of the simple protection garment of FIG. 9 configured as a backpack, and including a user's belt providing a detachable grounding strap.

FIG. 13 illustrates an exemplary helmet embodiment of a protective garment.

FIG. 14 illustrates another exemplary helmet embodiment of a protective garment.

FIG. 15 illustrates another exemplary helmet embodiment of a protective garment.

List of Reference Symbols in Drawing Figures

| Ref. | |
|---|---|
| Symbol | Element Name |
| 100 | Lightning cardiopulmonary protection garment, generally |
| 102 | Garment body (including a conductive body shield) |
| 104 | Garment hood (including a portion of the conductive shield) |
| 106 | Garment pocket |
| 108 | Grounding strap |
| 110 | Weighted end |
| 112 | Local ground plane |
| 200 | Garment cloth including electrically conductive layer, |
| | generally |
| 202 | Electrically conductive layer |
| 204, 206 | Cloth layer |
| 300 | Lightning cardiopulmonary protection garment, generally |
| 302 | Electrically conductive body shield |
| 304 | Reduced conductivity heart-protectionregion |
| 306 | Reduced conductivity material |
| 308 | Garment hood (including portion of conductive shield) |
| 400 | Details ofelectrical connection, generally |
| 402 | Electrically conductive layer |
| 404 | Ground strap |
| 406 | Electrical connection |
| 408 | Weighted end |
| 410, 412 | Cloth layers |
| 500 | Alternative ground strap configuration, generally |
| 502 | Conductive shield material |
| 504 | Electrical connection |
| 506 | Ground strap |
| 508 | Weighted end |
| 600 | Conductive body shields with reduced conductivity regions, |
| | generally |
| 602, 606, | Conductive shield |
| 610, 614 | |
| 604, 608, | Low-conductivity region |
| 612, 616 | |
| 700 | Lightning cardiopulmonary protection garment, generally |
| 702 | Garment body (including a conductive body shield) |
| 704 | Garment pocket |
| 706 | Grounding strap |
| 708 | Shoe |
| 709 | Heel and sole of shoe |
| 710 | Local ground plane |
| 712 | Lower end of grounding strap |
| 714 | Attachment to heel of shoe |
| 716 | Alternative grounding strap |
| 718 | Tail end of grounding strap |
| 720 | Lower end of alternative grounding strap |
| 800 | Protective garment, generally |
| 802 | Protective jacket |
| 804 | Jacket hood |
| 806 | Protective pants |
| 808 | Electrically conductive fabric strip (back) |
| 810 | Electrically conductive fabric strip(arms, shoulders) |
| 812 | Electrically conductive fabric strip (bottom of jacket) |
| 814 | Electrically conductive fabric strip (legs) |
| 816 | Example cutaway illustrating inner heat shielding layer |
| 818 | Example cutaway illustrating inner heat shielding layer |
| 820 | Example cutaway illustrating inner heat shielding layer |
| 822 | Upper back electrical junction, generally |
| 824 | Lower back electrical junction, generally |
| 826 | Strap electrically connecting jacket and pants, generally |
| 828 | Ground discharge ring |
| 830 | Electrically conductive connection strap |
| 832 | Removable strap fastener |
| 900 | Alternative embodiment, generally (cape) |
| 902 | Wearable moisture absorbent fabric cape |
| 904 | Electrically conductive fabric strip (back) |
| 906 | User's waist |
| 1000 | Alternative embodiment, generally (vest) |
| 1002 | Wearable moisture absorbent fabric vest |
| 1004 | Electrically conductive fabric strip (back) |
| 1100 | Alternative embodiment, generally (shirt, rear view) |
| 1102 | Posterior side |
| 1104 | Detachable, electrically conductive element |
| 1106 | Attachment means |
| 1108 | Attachment means |
| 1110 | Attachment means |
| 1112 | Detachable, electrically conductive belt |
| 1114 | Non-buckle end |
| 1116 | Buckle end |
| 1150 | Alternative embodiment, generally (shirt, side view) |
| 1200 | Alternative embodiment, generally (backpack, rear view) |
| 1202 | Electrically conductive fabric portions |
| 1204 | Shoulder harness |
| 1206 | Waist strap |
| 1208 | Sliding fastener |
| 1210 | Sliding fastener |
| 1212 | Sliding fastener |
| 1214 | Sliding fastener |
| 1216 | Detachable, electrically conductive belt |
| 1218 | Non-buckle end |
| 1220 | Buckle end |

### DESCRIPTION OF THE PREFERRED EMBODIMENT

With reference to FIG. 1, there is shown a pictorial side view illustrating a cardio-pulmonary lightning protection garment providing a flashover promoting, electrically conductive body shield (not illustrated) and a grounding strap with weighted end. The protective garment is designated generally by reference numeral 100, and includes a garment body 102, a hood 104, pocket 106, and a grounding strap 108 with weighted end 110 in contact with a local ground plane 112. When not in use, grounding strap 108 and weighted end 110 are carried within pocket 106.

In a specific embodiment, garment 100 is manufactured out of cloth so that it appears to be normal clothing, specifically, a hooded jacket as shown in FIG. 1. In this embodiment, the textile out of which the jacket hood, body, and sleeves are constructed includes a flashover promoting, electrically conductive body shield having a heart protection region located at the chest area adjacent to the wearer's heart. Grounding strap 108 is electrically connected at its upper end to the conductive body shield, and in the event of a lightning strike, carries the electrical charge away from the heart, across the shield, and to the local ground plane 112. Weighted end 110 ensures that the grounding strap remains in a sliding contact with local ground plane 112 as the wearer moves about.

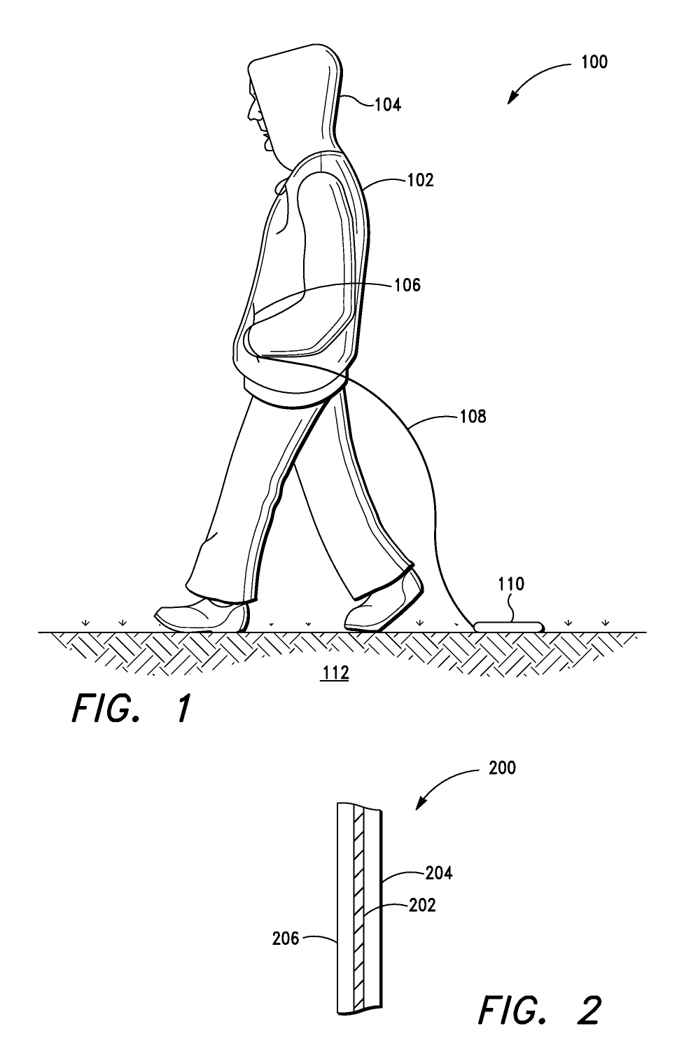

FIG. 2 is a partial side view that illustrates an electrically conductive layer enclosed between cloth layers for use in manufacture of the protective garment of FIG. 1. The specific construction detail is designated generally by reference numeral 200, and includes electrically conductive layer 202 sandwiched between outer cloth layers 204, 206.

Various forms of conductive layer 202 are contemplated including enclosure between cloth layers, as shown in FIG. 2 and taught in U.S. Pat. No. 7,284,280, and as an intermediate layer of the garment as taught in U.S. Pat. No. 6,272,781. Other arrangements with and without additional cloth layers include a textile fabric with integrated electrically conductive fibers as taught in U.S. Pat. No. 5,906,004; a nano-reinforced carbon fiber composite material as taught in U.S. Pat. No. 7,832,983; and a metallic nano-strand conductive composite material as taught in U.S. Pat. No. 7,947,773. The teachings of each of the US patents cited above are incorporated herein by reference.

In another specific embodiment, the garment is manufactured from a sandwiched construction such as illustrated in FIG. 2. The electrically conductive layer 202 forms a flashover promoting, electrically conductive body shield by having seams of the garment join adjacent parts of the conductive layer 202 so that the resulting garment forms an electrically conductive entity.

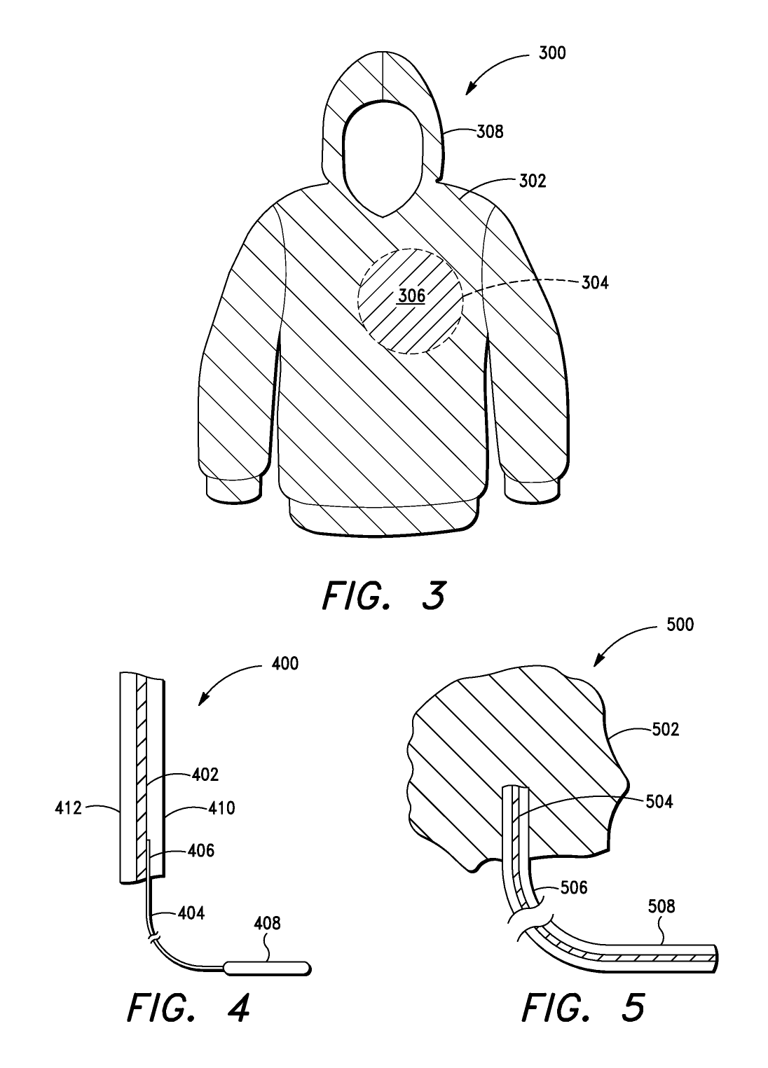

FIG. 3 is a partial front view illustrating a region of the conductive body shield having a reduced conductivity in the vicinity of the heart. The details of the body shield and the reduced conductivity region are designated generally by reference numeral 300 and include an electrically conductive body shield 302 having a reduced conductivity heart-protection region 304 covered by a reduced conductivity material 306, and garment hood 308 also including a portion of the body shield 302.

The body shield 302 is made of the electrically conductive layer 202 of FIG. 2 and, in the specific embodiment illustrated in FIG. 3, encloses the torso body, hood and sleeves of the garment 300. The various parts, from which the body shield is constructed, are connected at the construction seams to form an electrically conductive whole. A region adjacent to the wearer's heart (circular area 304 inside the dashed line at the left side of the chest) is made of a material 306 having a reduced electrical conductivity as compared with the conductivity of the remainder of the body shield 302. In some embodiments the body shield 302 has a first level of conductivity while the heart-protection region 304 has a second level of conductivity-a region of reduced conductivity. In other embodiments, the region 304 near the heart is a void in the body shield, and thus has zero conductivity. The purpose of the heart-protection region is to direct the intense lightning charge of short duration away from the heart, without interfering with the promotion of flashover, to prevent or lessen the chance of cardiopulmonary arrest.

FIG. 4 is a partial side view illustrating an electrical connection between a conductive body shield and a grounding strap made of a flexible conductor, and illustrating a weighted end. Details of the electrical connection are designated generally by reference numeral 400, and include electrically conductive layer 402, ground strap 404, electrical connection 406, weighted end 408, and cloth layers 410, 412. The electrical connection between the conductive layer 402, used to form the conductive body shield, and the ground strap 404, completes an electrical circuit permitting the ground strap 404 to carry the lightning charge from the body shield down to ground (112 of FIG. 1).

FIG. 5 is a partial plan view that illustrates an alternative configuration for a grounding strap made of a cloth-covered extension of the conductive body shield, and illustrating a weighted end. Details of the alternative grounding strap configuration are designated generally by the reference numeral 500, and include conductive shield material 502, electrical connection 504, ground strap 506, and weighted end 508. This configuration replaces the flexible conductor 404 of FIG. 4 with a cloth strap having an internal electrical conductor such as the conductive layer 202 of FIG. 2. The strap and its weighted end are carried in a garment pocket when not in use. An alternative embodiment allows the weighted end 508 to be attached inside the garment using Velcro®, an ordinary button, or like attachment (not illustrated), e.g., attached up inside to a garment inner lining. In general, non-metallic fasteners are preferred because of the extreme voltages present during a lightning strike.

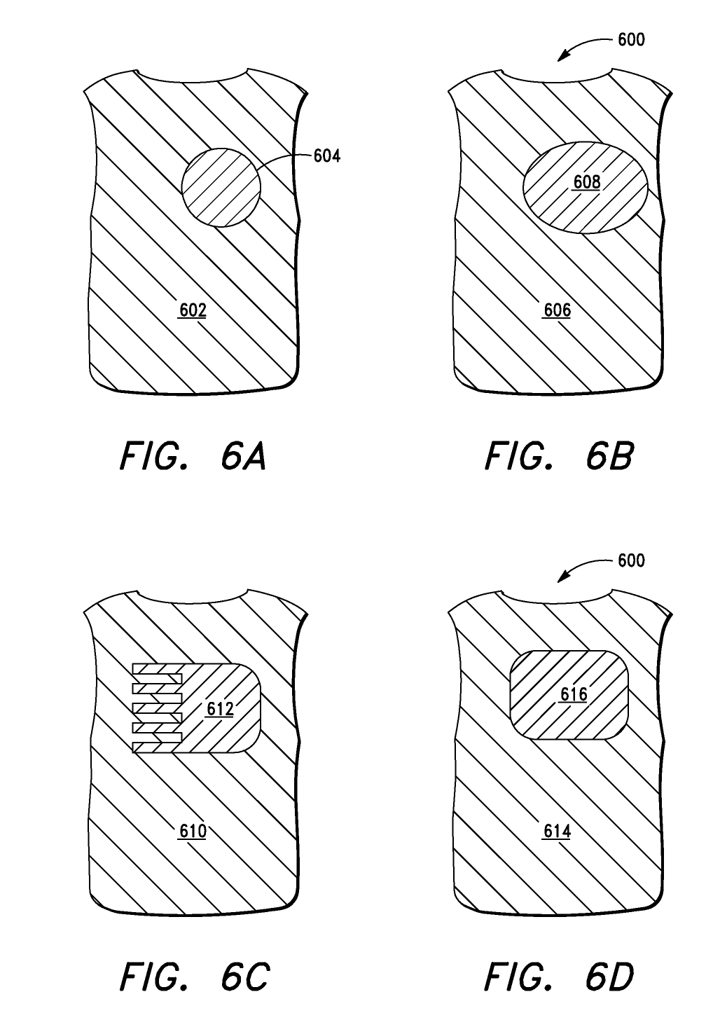

FIGS. 6A-6D illustrate alternative arrangements of a low-conductivity region near the heart. The figures illustrate a front chest region of a body shield and region of low conductivity. The various low-conductivity regions are designated generally by reference numeral 600 and include high-conductivity shields 602, 606, 610, and 614, and regions of low conductivity 604, 608, 612, and 616, respectively.

One variation in the regions of low-conductivity is that they are of different size and shape. In particular, the region 612 in FIG. 6C is open adjacent to the wearer's heart on the center-left side of the chest area and includes rib-like horizontal extensions of the conductive body shield 610 on the right side of the chest.

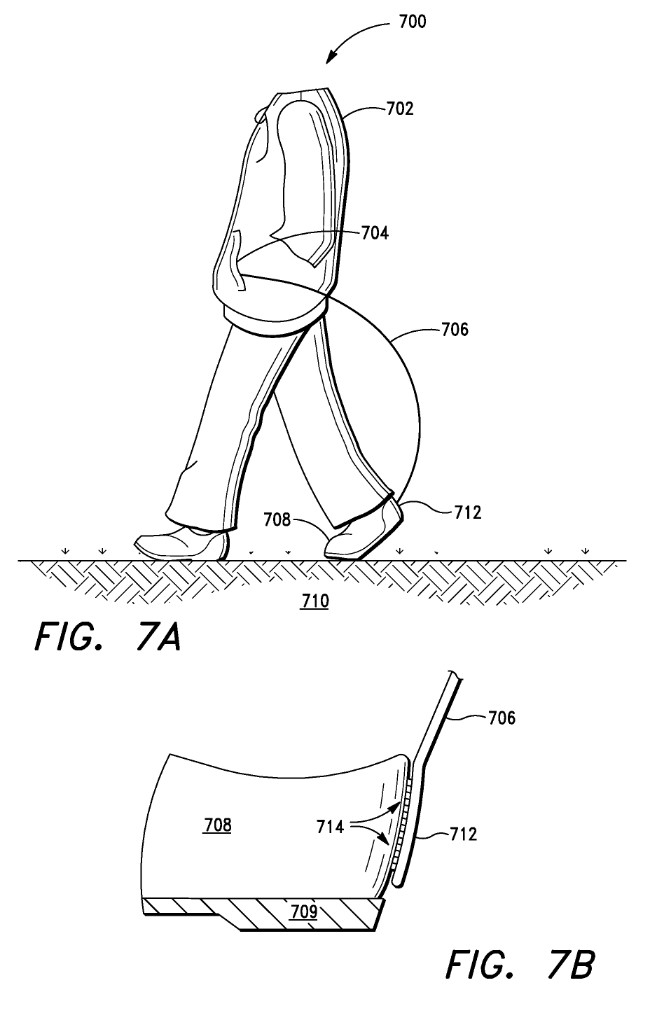

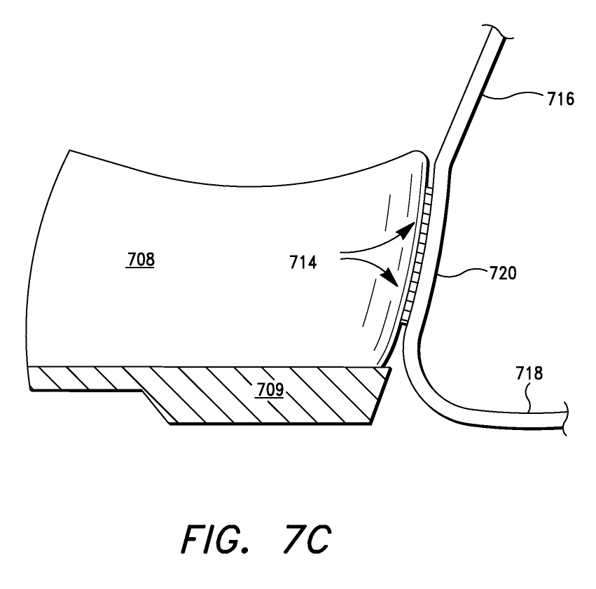

FIGS. 7A-7C illustrate an alternative construction for the lower end of the grounding strap, that is here shown attached to the heel of the shoe rather than having a weighted lower end. FIG. 7A illustrates a lightning cardiopulmonary protection garment that is indicated generally by the reference numeral 700, and includes a garment 702 having a conductive body shield (not illustrated), a garment pocket 704, an alternative grounding strap 706 attached 712 to the heel of shoe 708, and illustrating a local ground plane 710.

FIG. 7B is a partial side view of shoe 708 of FIG. 7A showing heel and sole 709, and a lower end 712 of alternative grounding strap 706 attached to the heel region of shoe 708 by means of attachment 714, such as Velcro®. Various embodiments of attachment 714 are not electrically conductive, for example, the Velcro® attachment. Nor, in general, are the heel and sole 709 of the shoe conductive. Other embodiments of shoes having electrically conductive heels, soles, and/or metal spikes, such as golf shoes (not illustrated), are also contemplated.

FIG. 7C illustrates an alternative grounding strap 716 including a tail portion 718 at the extreme lower end. In various embodiments, the grounding strap 716 is made of, or includes, electrically conductive material that extends into the region 718 and drag along the local ground plane (not illustrated). When spiked shoes are worn, the tail region 718 can be attached by pressing spikes near the rear region of heel 709 through a portion of the tail region 718 that is allowed to extend under the heel of the shoe (not illustrated). In another variation, the conductive grounding strap 706 connecting the body shield with the heel of shoe 708 is doubled, one strap for each shoe, to distribute the charge equally between both legs (FIG. 7A). In another variation, a wire or other conductive strap connects the heels of both shoes (not illustrated).

In specific embodiments of the protective shield (e.g., FIGS. 6A-6D), the region of low-conductivity (e.g., 604 of FIG. 6A) is made of a material having a significantly lower conductivity than the surrounding protective shield (602). The purpose of the shield is to promote a fast flashover effect, and to collect the electrical charge and direct it away from the region of the heart and to the grounding member (e.g., 108 of FIG. 1) making contact with a local ground plane (Earth), either directly or through the shoe (FIG. 7A). Use of a material of low conductivity in the region near the heart helps direct the charge away from the heart. In various embodiments that use a low-conductivity material in the heart region, the electrical connection between the high-conductivity protective shield and the low-conductivity heart protection region is a high-conductivity connection (not illustrated). In other embodiments, the low-conductivity region is created as a conductive void in the material of the protective shield. The phrase “conductive void” as used here refers to a hole—an absence of conductive material in the body shield. The cloth, of course, remains covering the heart region so that the garment appears natural. In another variation (not illustrated) the body shield does not include a region of reduced conductivity near the heart, and relies instead upon the fast flashover effect and electrical body shielding for protection.

### NEW EMBODIMENTS

Electrically conductive fabrics are costly, and thus a garment that includes a significant amount of conductive fabric, as in the electrically conductive body shield 302 of FIG. 3, may be priced beyond the reach of most users. Some lightning morbidity research indicates that persons who survive a lightning strike, despite first degree burning of the skin, have sometimes been protected by an ionization of the air near the surface of the body in what is called a “flashover” event. It appears that during such an event, the lightning travels through the ionized layer, producing the burns, rather than deeper through the body where it would likely disrupt normal heart function: the heart is protected, the victim survives. The research also teaches that it is the “rapid” flashover from which patients survive, not an ionizing event of longer duration or taking longer to reach a peak intensity. For example, a significant component of the lightning strikes studied by Ohashi et al. were continuing strikes of 20-400 milliseconds duration. The researchers concluded that victims do not survive such prolonged events. (Ohashi et al., pages 500, 501).

The new embodiments presented here take advantage of this research by attempting to provoke a rapid flashover event in the presence of an intense electrical field-lightning. The electrically conductive body shield of FIG. 3 is replaced by a wet or dampened fabric garment that will support an ionizing (flashover) event. In other words, once flashover is initiated, it will take place in an ionized layer near the outer surface of a wet or dampened garment constructed to protect the user to the extent possible.

Most of the ionized layer is encouraged to occur at or near an outer surface of a waterproof/breathable barrier. Strips of electrically conductive fabric, placed, for the most part, on an outer posterior surface of the garment, are added to ignite and to promote a rapid ionizing event (flashover). The conductive strips placed at the back of the protective garment are shown to be effective for this purpose, and of equal importance, tend to steer the flashover toward the back and away from the chest area of the body and the heart. In some embodiments, an inner layer of heat and fire resistant material, such as NOMEX® and equivalents, is provided for protection from the most severe burning.

An embodiment of a cardiopulmonary lightning protection garment includes an outer waterproof/breathable raincoat that becomes wet and supports an ignited flashover. The word raincoat used here includes such things as a rain jacket and the like. The raincoat can be made of any waterproof/breathable fabric such as, for example, Gore-Tex® laminates and their equivalents. A strip of an electrically conductive fabric, such as Shieldex® and equivalents, is placed along the spine region on the outer surface of the raincoat. The strip serves to promote ignition of a rapid flashover that then forms an ionized layer surrounding the wet surface of the raincoat, conducting the largest part of the electrical charge of the lightning to the surface and away from the trunk of the user's body. To reduce injury from burning, an inner layer of a heat shielding/fire resistant fabric, such as NOMEX® and equivalents, can be provided. As stated earlier, a user is likely to be seriously injured as a result of a lightning strike, and the flashover, when that occurs. Some users will die as an immediate result or following injury. But the flashover, when it occurs, will save the lives of some users. Some embodiments include various forms of grounding strap for carrying the electrical charge away from the user to a local ground plane, such as the Earth, as illustrated in FIGS. 1, 4, and 7A-7C.

It is believed that placing an electrically conductive fabric strip on the posterior side of the garment, in general alignment with a wearer's spine, is the optimum configuration. However, one or more strips placed in other locations of the outer surface of a garment can still promote ignition of the rapid flashover event. Thus, such other configurations are also contemplated for use in some embodiments of these protective garments.

### EXAMPLE EMBODIMENTS

What follows is a collection of example embodiments, each designated E_, providing additional description of a variety of embodiment types in accordance with the concepts described herein. These examples are not meant to be mutually exclusive, exhaustive, or restrictive, and the invention is not limited to these example embodiments, but rather encompasses all possible modifications and variations within the scope of the issued claims.

E1. An article of manufacture, comprising:
a. a waterproof-breathable fabric garment for covering at least a portion of a wearer's body, for supporting a flashover when the garment is damp; and
b. an electrically conductive element, located on the garment for encouraging a rapid flashover event.
   E2. The article of manufacture of embodiment E1, wherein the garment covers at least the upper portion of the wearer's body and extends at least to the wearer's waist.
      E3. The article of manufacture of embodiment E2, wherein the garment extends at least to the wearer's hips.
      E4. The article of manufacture of embodiment E2, wherein the garment extends at least to the wearer's knees.
E5. The article of manufacture of embodiment E1, wherein the waterproof-breathable fabric is a Gore-Tex® laminate, or equivalent.
E6. The article of manufacture of embodiment E1, wherein the electrically conductive element is a strip of electrically conductive fabric, such as Shieldex®, or equivalent.
E7. The article of manufacture of embodiment E1, wherein the electrically conductive element is a metal slide fastener.
E8. The article of manufacture of embodiment E1, further comprising a grounding member connected to the electrically conductive element for conducting electrical charge to a local ground plane to sustain rapid flashover.
   E9. The article of manufacture of embodiment E8, further comprising the grounding member establishing a movable electrical connection between the electrically conductive element and a ground plane, such as the Earth.
E10. The article of manufacture of embodiment E1, further comprising a protective heat shield layer formed of a material such as NOMEX®, or equivalent.
   E11. The article of manufacture of embodiment E6, wherein the strip of electrically conductive fabric extends from an upper region to a lower region of the posterior side of the garment.
      E12. The article of manufacture of embodiment E11, further comprising a conductive strip located horizontally at a lower region of the posterior side of the garment.
         E13. The article of manufacture of embodiment E12, wherein the conductive strips are electrically connected.
E14. The article of manufacture of embodiment E1, wherein the garment comprises a jacket having sleeves and opening at the anterior side of the garment.
   E15. The article of manufacture of embodiment E14, further comprising conductive strips located on the posterior side of the jacket.
      E16. The article of manufacture of embodiment E15, further comprising conductive strips located on a lateral surface of the sleeves, away from the trunk of the wearer's body, all conductive strips being joined at one or more electrically conductive intersections.
E17. The article of manufacture of embodiment E1, further comprising any arrangement of conductive fabric located on the posterior side of the garment, wherein parts of the arrangement are electrically connected.
   E18. The article of manufacture of embodiment E17, an arrangement includes a full covering of the posterior side of the garment.
E19. The article of manufacture of embodiment E1, wherein the electrically conductive material further comprises a textile fabric with integrated electrically conductive fibers.
E20. The article of manufacture of embodiment E1, wherein the electrically conductive material further comprises a nano-reinforced carbon fiber composite material.
E21. The article of manufacture of embodiment E1, wherein the electrically conductive material further comprises a metallic nano-strand conductive composite material.
E22. The article of manufacture of embodiment E1, wherein the article comprises one of a jacket, coat, suit coat, top coat, sweater, vest, sweatshirt, raincoat, and poncho.
   E23. A cardiopulmonary protection garment for providing a wearer with limited protection from lightning, comprising:
a. an outer layer forming a partial body covering of a waterproof/breathable fabric, such as a Gore-Tex® laminate or equivalent, for keeping the wearer dry and for supporting a flashover event when wet or damp and struck by lightning; and
b. an electrically conductive rapid flashover igniting means located upon an outer surface of the body covering.
E24. The cardiopulmonary protection garment of embodiment E23, wherein the body covering further comprises an inner heat shielding and flame resistant fabric layer, such as NOMEX® or equivalent, for reducing burn injuries.
E25. The cardiopulmonary protection garment of embodiment E23, further comprising ground plane connection means, electrically connected with the conductive rapid flashover igniting means for carrying lightning charge to a local ground plane, such as the Earth.
E26. The cardiopulmonary protection garment of embodiment E23, wherein the body covering takes the form of one of a rain jacket, a hooded rain jacket, a long raincoat, a hooded long raincoat, a rain jacket and rain pants, a hooded rain jacket and rain pants, a one-piece waterproof coverall, a hooded one-piece waterproof coverall, a padded vest, and a rain poncho.
   E27. The cardiopulmonary protection garment of embodiment E25, wherein when the garment takes the form of separate jacket and pants, both the jacket and pants include a portion of the electrically conductive rapid flashover igniting means.
      E28. The cardiopulmonary protection garment of embodiment E27, wherein the separate portions of the electrically conductive rapid flashover igniting means are electrically connected to each other.
E29. The cardiopulmonary protection garment of embodiment E23, wherein the electrically conductive rapid flashover igniting means is a metal slide fastener.
E30. The cardiopulmonary protection garment of embodiment E23, wherein the electrically conductive rapid flashover igniting means further comprises a drape that hangs from the shoulders over the waterproof/breathable layer and wherein at least a portion of the posterior side of the drape includes an electrically conductive fabric.
   E31. The cardiopulmonary protection garment of embodiment E30, wherein the drape is in contact with the waterproof/breathable layer and is able to absorb moisture from the outer surface of the waterproof/breathable layer.
   E32. The cardiopulmonary protection garment of embodiment E30, wherein the electrically conductive rapid flashover igniting means further comprises a metal slide fastener.
      E33. A cardiopulmonary protection garment for providing a wearer with limited protection from lightning, comprising:
a. a wearable, water-absorbing fabric article; and
b. an electrically conductive element located on the article for igniting a rapid flashover.
E34. The cardiopulmonary protection garment of Embodiment E33, wherein the wearable, water-absorbing article is one of a cape-like fabric element, a jacket, a shirt, a sweatshirt, a hooded-sweatshirt, a sweater, and the like.
E35. The cardiopulmonary protection garment of Embodiment E33, wherein the electrically conductive element is detachable from the article, and including means for attaching the electrically conductive element to the article.
E36. The cardiopulmonary protection garment of embodiment E33, wherein the electrically conductive element is a metal slide fastener.
E37. The cardiopulmonary protection garment of Embodiment E33, further including a detachable grounding strap electrically connected to the electrically conductive element for conducting electrical charge to a local ground plane.
   E38. The cardiopulmonary protection garment of Embodiment E37, wherein the detachable grounding strap is an electrically conductive belt having a buckle end and a non-buckle end, the non-buckle end being adapted for attachment to the garment's electrically conductive element, permitting the buckle end to extend toward the local ground plane, or alternatively, to make contact with the local ground plane.
      E39. A cardiopulmonary protection backpack for providing a user with limited protection from lightning, comprising:
a. a backpack having an outer covering, at least a portion of which is made of a water-absorbing fabric; and
b. an electrically conductive element located on the backpack for igniting a rapid flashover.
E40. The cardiopulmonary protection backpack of Embodiment E39, wherein the backpack further includes a waterproof inner layer for maintaining a dry backpack interior.
E41. The cardiopulmonary protection backpack of Embodiment E39, wherein the electrically conductive element is detachable from the backpack, and including means for attaching the electrically conductive element to the backpack.
E42. The cardiopulmonary protection backpack of Embodiment E39, further including a detachable grounding strap, having means for electrical connection to the electrically conductive element, for conducting electrical charge to a local ground plane.
   E43. The cardiopulmonary protection backpack of Embodiment E42, wherein the detachable grounding strap is an electrically conductive belt having a buckle end and a non-buckle end, the non-buckle end being adapted for attachment to the garment's electrically conductive element, permitting the buckle end to extend toward the local ground plane, or alternatively, to make contact with the local ground plane.
      E44. A cardiopulmonary protection garment for providing a wearer with limited protection from lightning, comprising a wearable article made of a fabric including electrically conductive fibers for igniting a rapid flashover.
E45. The cardiopulmonary protection garment of embodiment E44, wherein the electrically conductive fibers are made of a noble metal such as silver.
E46. The cardiopulmonary protection garment of embodiment E45, wherein the fabric is a member of the Silverescent® family of moisture-wicking fabrics.
E47. The cardiopulmonary protection garment of embodiment E45, wherein the fabric is contoured to avoid the wearer's heart region.
E48. The cardiopulmonary protection garment of embodiment E45, wherein the electrically conductive fabric layer is combined with at least one non-electrically conductive fabric layer to improve appearance and wearability.

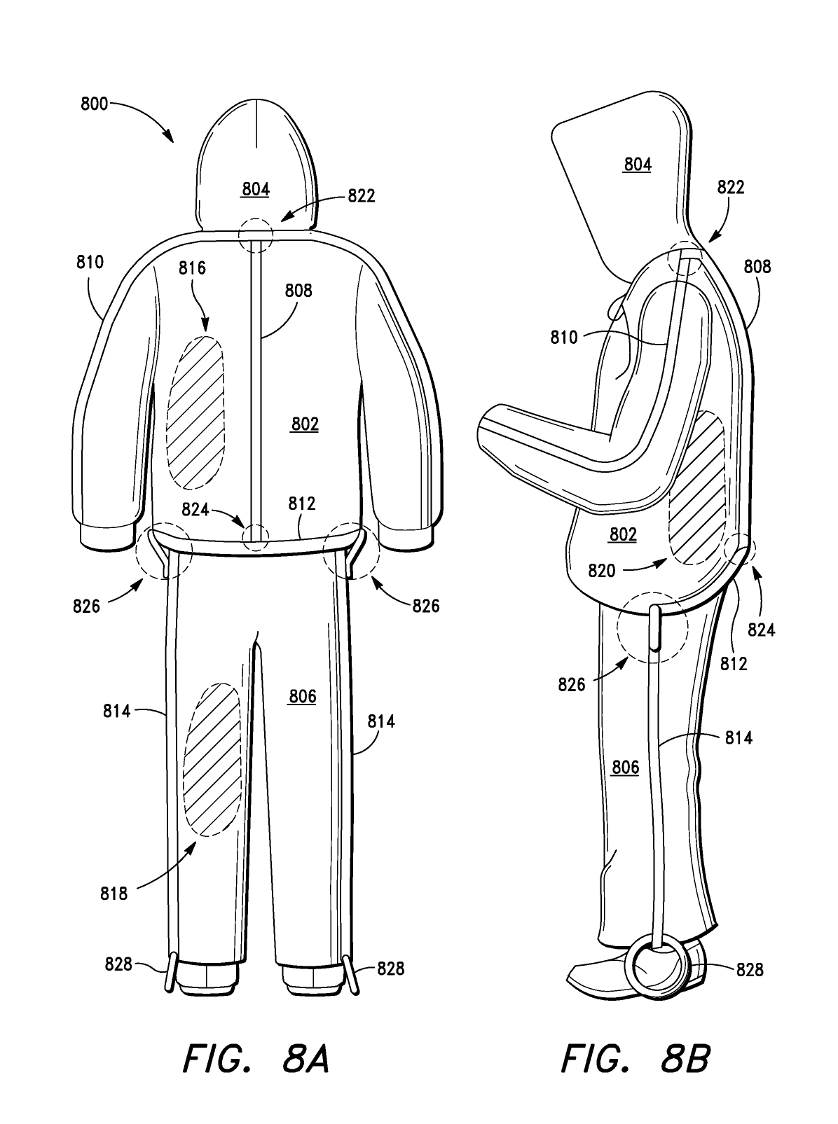

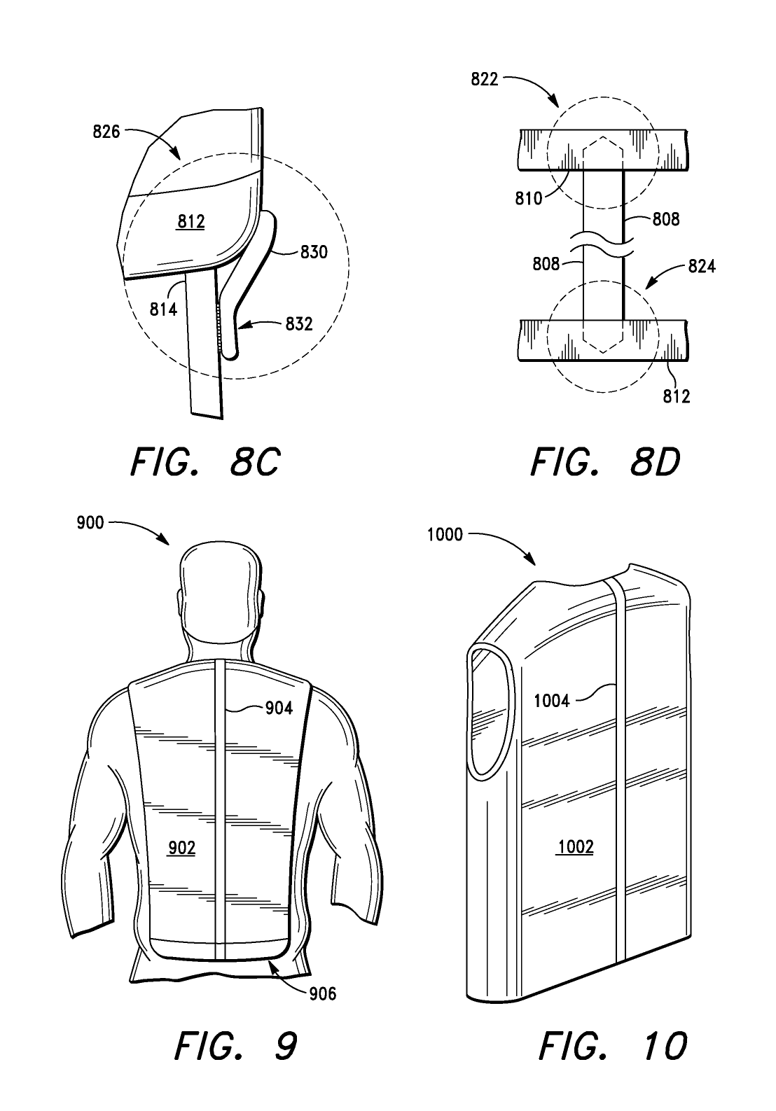

FIGS. 8A, 8B are rear and left side views, respectively, illustrating an embodiment of a cardiopulmonary lightning protective garment, designated generally by reference numeral 800 and including a hooded jacket and pants. The protective garment 800 includes protective jacket 802, jacket hood 804, and protective pants 806, all made of a waterproof/breathable fabric such as Gore-Tex® or equivalent. Conductive strips of an electrically conductive fabric, such as Shieldex® or equivalent are located at the lateral and posterior outer surface of the protective garment and include electrically conductive fabric strips 808, parallel to the spine, 810, along both sleeves and across the shoulders, 812, at the side and rear lower edge of protective jacket 802, and 814, along the lateral edge of both legs, respectively. Note that when a same or similar object appears in more than one view, it is given the same reference number. Thus, for example, electrically conductive fabric strip 812 at the lower edge of the protective jacket is given the same reference number in both FIGS. 8A and 8B. The electrically conductive fabric strips are not placed upon jacket hood 804 nor on the front side of protective jacket 802, thus providing some protection for the head and heart region by steering a flashover toward the posterior side. Electrically conductive fabric strip 808 is joined at its upper end to a mid-point of electrically conductive fabric strip 810 (the junction of the two strips indicated generally by broken line circle having reference number 822), and at its lower end to a mid-point of electrically conductive fabric strip 812 (indicated generally by broken line circle and reference number 824). The forward lateral ends of electrically conductive fabric strip 812 can be connected, for example, by straps (indicated generally by reference numbers 826, left and right, and more particularly illustrated by FIG. 8C).

In some embodiments (not illustrated), protective garment 800 does not include protective pants 806, while in other embodiments (also not illustrated) protective jacket 802 includes a detachable jacket hood 804 and, in some embodiments, includes no hood at all. In some embodiments (also not illustrated), electrically conductive fabric strip 808 is replaced by a metal slide fastener, such as a Zipper®.

In yet other embodiments, protective garment 800 includes an inner heat shielding layer made of a fire resistant fabric such as NOMEX® or equivalent. This additional protective layer is placed beneath the outer waterproof/breathable layer. The presence of such an inner heat shielding layer is illustrated in FIGS. 8A and 8B by the shaded areas, designated generally as 816-820 and encircled by broken lines. In an embodiment, the heat shielding layer is included in protective jacket 802, jacket hood 804, and protective pants 806. In some embodiments, additional fabric layers are added for providing warmth and/or comfort.

Persons who have survived lightning strikes show burns on the skin of the body's trunk. In embodiments that include separate protective pants, as shown in FIGS. 8A, 8B, electrically conductive fabric strips 814 can be provided along the lateral side of each leg. FIGS. 8A, 8B show a metallic ground discharge ring 828 attached at the bottom of the electrically conductive fabric strips 814 along the pant legs. The ground discharge rings 828 are electrically connected (e.g., by electrical contact) with the lower end of each pant leg strip, and are located near the ground when the user is standing, as shown in FIGS. 8A, 8B. The ground discharge rings 828 provide a discharge path to a local ground plane, such as the Earth.

All electrically conductive fabric strips are joined electrically at crossing points, for example at points indicated by the broken circles 822, 824, and 826. In particular, when separate protective pants and jacket are worn, as illustrated in FIGS. 8A, 8B, a small electrically conductive fabric strip 830 attached to electrically conductive fabric strip 812 can be used to connect the interconnected strips of the jacket with the strips 814 of the pants, and the discharge rings 828.

FIG. 8C illustrates details of an embodiment 826 using the electrically conductive fabric strap 830 for electrically connecting the conductive strip 812 with the conductive strips 814 of the protective pants 806. The broken line circle 826 corresponds to broken line circle 826 in FIGS. 8A, 8B. In the embodiment illustrated, strap 830 is permanently attached to jacket conductive strip 812, and is can be connected to the pants legs strip 814 by the user. An attachment 832 such as Velcro® can be used for attaching strap 830 to electrically conductive fabric strip 814.

FIG. 8D illustrates details of the electrical connection between intersecting electrically conductive fabric strips shown in FIGS. 8A, 8B. Broken circle 822 highlights an intersection between electrically conductive fabric strip 810 (jacket sleeves and shoulders) with conductive strip 808 (parallel with user's spine), and corresponds with broken circle 822 of FIGS. 8A, 8B. Broken circle 824 highlights an intersection between electrically conductive fabric strip 812 (bottom of jacket) with conductive strip 808 (parallel with user's spine), and corresponds with broken circle 824 of FIGS. 8A, 8B.

In other embodiments, a grounding element is added providing an electrical path from the wet garment and conductive strips to a local ground-plane, such as the Earth (see, for example, FIGS. 1, 4, and 7A-7C). The grounding element takes several forms including a wire, chain, or conductive fabric dragging along the ground, or attached to a weight to maintain contact with the ground. In another embodiment, the grounding element attaches to the wearer's shoe(s) to maintain close proximity with the ground. In an embodiment in which the garment includes trousers having legs, a conductive strip along one or both trouser legs electrically connects at an upper end with conductive strips on the upper part of the garment and at a lower end with metallic rings and in one embodiment, attachment to wearer's shoe(s), and in another embodiment with a weighted, sliding ground-contacting member. When such alternative grounding elements are used, the ground discharge rings 828 may still be present.

Waterproof/breathable fabrics resist liquid water passing through, but allow water vapor to pass through. Their ability to block out rain and snow while allowing vapor from sweat to evaporate leads to their use in rainwear, waterproof outdoor sports clothing, tents, and other applications. Standard laboratory testing protocols define the performance of these fabrics. Water resistance is measured by the amount of water, in mm, which can be suspended above the fabric before water seeps through. Breathability or moisture vapor transmission rate is measured by the rate at which water vapor passes through, in grams of water vapor per square meter of fabric per 24 hour period (g/m2/d), often abbreviated to just “g”. In recent years some, but not all, sporting goods manufacturers have begun including this information on their product labels. Typical mid-range fabrics tend to have values of 5,000 mm of water resistance and 5,000 g of breathability; the best materials have 20,000 mm and 20,000 g. One specific definition of “waterproof/breathable” requires the fabric to withstand over 1,000 millimeters of water (9.8 kPa) pressure without leaking (see hydrostatic head).

Gore-Tex® materials are typically based on thermo-mechanically expanded polytetrafluoroethylene (PTFE) and other fluoropolymer products. They are used in a wide variety of applications such as high performance fabrics, medical implants, filter media, insulation for wires and cables, gaskets, and sealants. However, Gore-Tex fabric is best known for its use in protective, yet breathable, rainwear.

NOMEX® is a registered trademark for flame-resistant meta-aramid material developed in the early 1960s by DuPont and first marketed in 1967. Nomex and related aramid polymers are related to nylon, but have aromatic backbones, and hence are more rigid and more durable. Nomex is the premier example of a meta variant of the aramids (Kevlar is a para aramid). Unlike Kevlar, Nomex cannot align during filament formation and has poorer strength. However, it has excellent thermal, chemical, and radiation resistance for a polymer material. A Nomex hood is a common piece of racing and firefighting equipment. It is placed on the head on top of a firefighter's face mask. The hood protects the portions of the head not covered by the helmet and face mask from the intense heat of the fire.

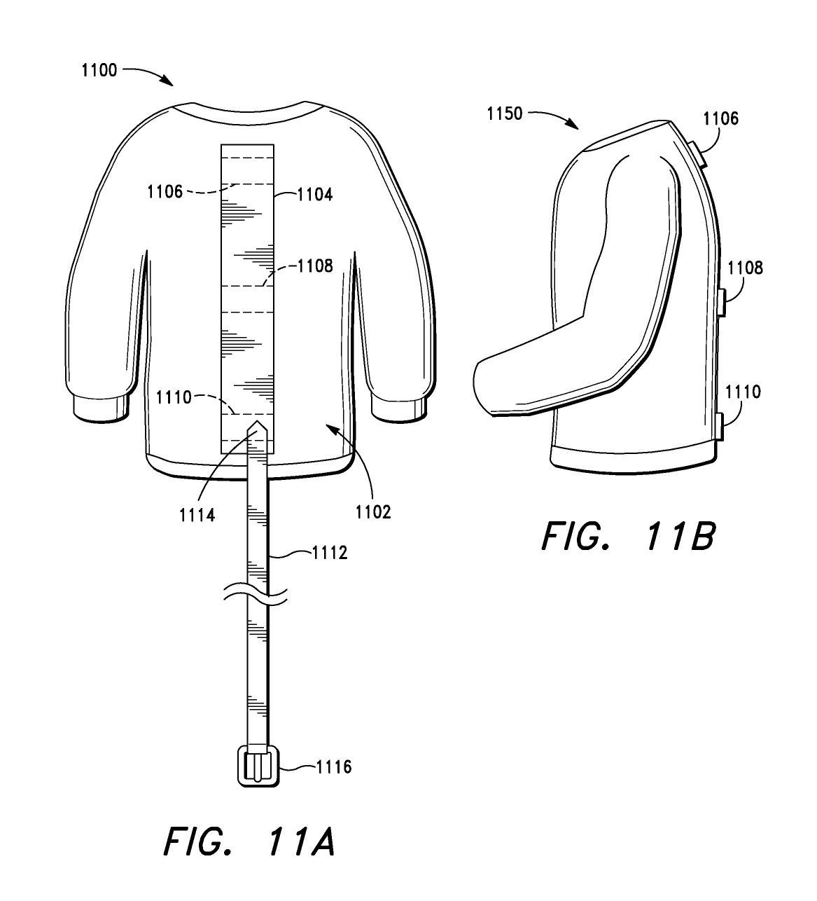

Shieldex® is a registered trademark for an electrically conductive metalized nylon fabric having low electrical resistance. The metalized layers are tin, nickel, and silver. The conductive fabric is foldable, flexible, and has good abrasion resistance. Shieldex, and like materials, are expensive. At the present time, there appears to be no low-cost electrically conductive fabric. This problem is overcome in the embodiments illustrated with respect to FIGS. 8A-8C, and 9 by recognizing that the moisture contained within a wet raincoat made of a waterproof/breathable material can support a flashover. Reliance on the rain garment permits the amount of electrically conductive fabric to be reduced from the body shield to rapid flashover igniting strips (e.g., 808, 810, 812, 814 of FIG. 8A).

FIG. 9 illustrates an alternative embodiment of a protective garment for use with separate raingear. The garment is depicted, generally, by reference numeral 900 and includes a cape-like fabric element 902 that lies along the back of a user and has a strip of electrically conductive fabric 904 attached to an outer surface in a vertical direction, along the user's spine. At the user's waist 906, a hold-down element (not illustrated) is provided for maintaining the garment aligned with the user's body. The protective garment 900 is worn over a separate raincoat or other rainwear (e.g., a rain poncho or jacket). The cape-like fabric element 902 is made of a water-absorbent fabric to aid in supporting a flashover. An advantage of this embodiment, and similar protective devices, is that it can easily be folded and stored in a pocket or backpack for ease of carrying when not in use. Though not providing the level of protection of embodiments such as those illustrated in FIGS. 8A, 8B (e.g., fewer conductive strips, no fire resistant inner layer), it relies upon the same principles of operation. In a variation of the embodiment of FIG. 9, a ground strap/wire such as illustrated in FIGS. 1, 4, and 7A-7C can be provided. When such a ground strap/wire is provided, its upper end is electrically connected to the lower end of electrically conductive strip 904. In some embodiments (not illustrated), electrically conductive strip 904 is replaced by a metal slide fastener.

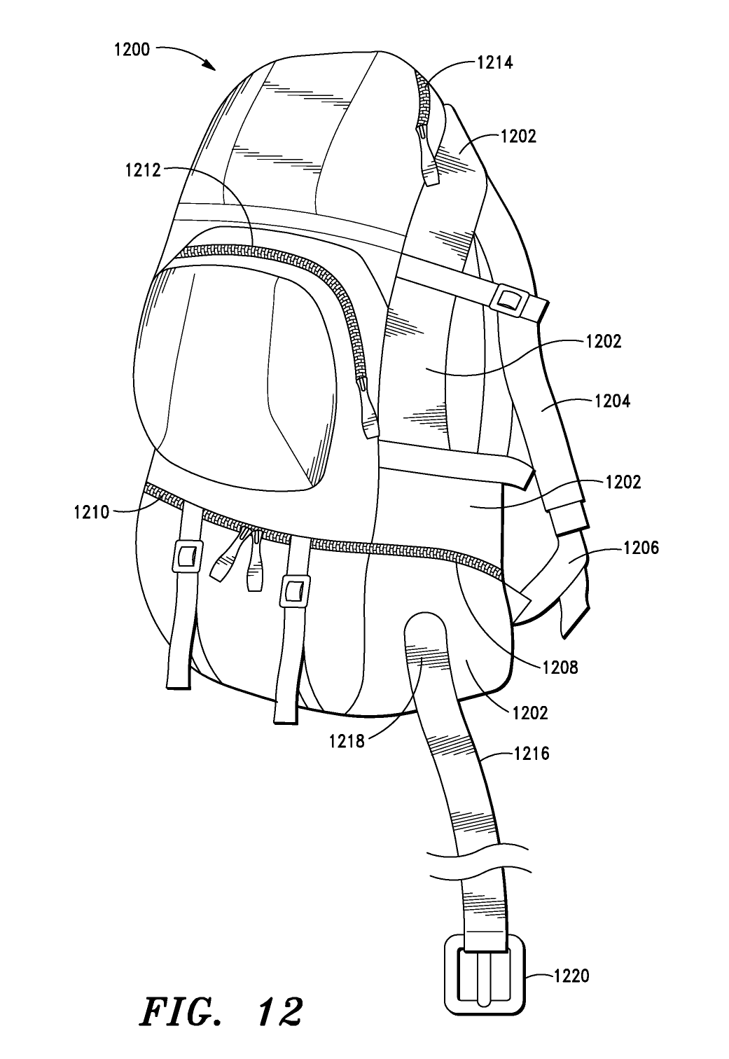

FIG. 10 illustrates another embodiment of a protective garment for use with separate rainwear. The protective garment is designated generally by reference number 1000 and includes a vest-like garment 1002 made of a moisture absorbent fabric and having an electrically conductive fabric strip 1004 attached at the rear surface parallel with the user's spine. In some embodiments (not illustrated), electrically conductive fabric strip 1004 is replaced by a metal slide fastener.

FIGS. 11A, 11B illustrate an alternative embodiment of the simple protection garment of FIG. 9 configured as a shirt, and including a user's belt providing a detachable grounding strap. FIG. 11A is a rear view, is designated generally by reference numeral 1100, and includes a posterior side 1102, a detachable, electrically conductive element 1104, attachment means 1106, 1108, and 1110, a detachable, electrically conductive belt 1112, having a non-buckle end 1114, and a buckle end 1116. FIG. 11B is a side view of the protective garment and includes the three attachment means 1106-1110. It will be understood that the shirt configuration shown in FIGS. 11A, 11B is illustrative only, and that other alternative embodiments of the simple protective garment of FIG. 9 include water-absorbent fabric constructions such as a jacket, a sweatshirt, a hooded-sweatshirt, a sweater, and the like. In some embodiments, the electrically conductive fabric element 1104 is permanently attached to the posterior side 1102. In other embodiments, electrically conductive fabric element 1104 is made a part of, the posterior side 1102, while in yet other embodiments is detachable from the garment and is attached when needed using a variety of common devices, such as the Velcro® attachments 1106-1110. The electrically conductive belt 1112 is normally worn or carried by a user, for example, to hold up trousers, but may be attached to the electrically conductive fabric element 1104 and allowed to extend the buckle end 1116 toward a local ground plane (not illustrated), or, depending upon the length of the belt and the height of the user, to permit the buckle end 1116 to be in a sliding contact with the local ground plane (see, for example, weighted end 110 of FIG. 1). In some embodiments (not illustrated), electrically conductive fabric element 1104 is replaced by a metal slide fastener that is incorporated into the garment. In other embodiments (also not illustrated), the metal slide fastener is attached to a posterior side of the garment in general alignment with a wearer's spine.

FIG. 12 is a rear-side view that illustrates an alternative embodiment of the simple protection garment of FIG. 9 configured as a backpack, and optionally including a user's belt, which may further provide a detachable grounding strap. The backpack is designated generally by reference numeral 1200 and may include electrically conductive fabric portions 1202, a shoulder harness 1204, a waist strap 1206, sliding fasteners 1208, 1210, 1212, and 1214, and a detachable, electrically conductive belt 1216 having a non-buckle end 1218 and a buckle end 1220. In some embodiments the backpack 1200 is made of a water-absorbent outer fabric for promoting rapid flashover, and has an inner waterproof lining for maintaining a dry backpack interior. At least a portion of the outer fabric includes electrically conductive portions 1202 for igniting the rapid flashover. In an embodiment, the electrically conductive portions 1202 are electrically connected to one another, such as by overlapping and joining (not illustrated). The optional electrically conductive belt 1216 is attachable at its non-buckle end 1218 to a portion 1202 of the electrically conductive fabric element, and extends from the backpack 1200 toward a local ground plane (not illustrated), or, depending upon the length of the belt 1216 and the height of the user, to be in a sliding contact with the local ground plane.

According to some exemplary embodiments, the surfaces of the backpack that touch or are adjacent to a user's body, when worn, may optionally have a lower conductivity or no conductivity. These surfaces may include the inner surfaces of the shoulder harness, waist strap, and the back panel, as would be understood by a person having ordinary skill in the art. This may direct the flashover away from a user's body. In some exemplary embodiments, a heat resistant layer or material may be disposed on surfaces touching or in close proximity to a user's body. The backpack may further be worn in combination with other embodiments of protective garments, as would be understood by a person having ordinary skill in the art. The optional electrically conductive belt may further contact or otherwise couple the grounding strap or conductive material of another protective garment worn by the user. In some embodiments, the conductive material disposed on the backpack may further include nanotechnology, such as metallic nano-strand conductive composite material or nano-reinforced carbon fiber composite material. However, other embodiments do not utilize nanotechnology in the conductive material.

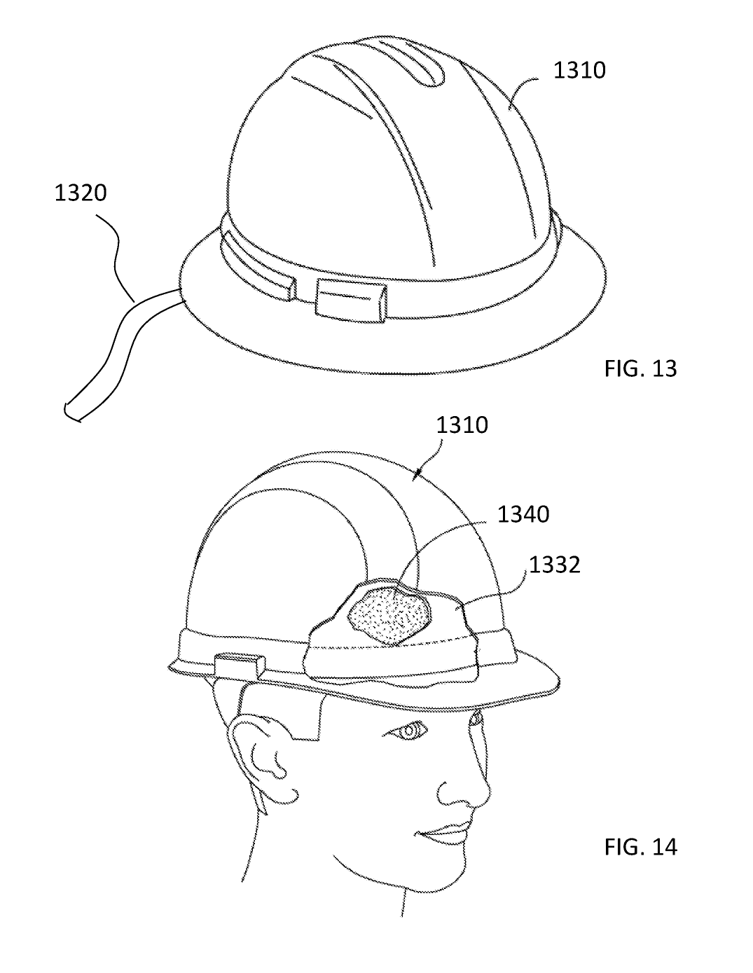

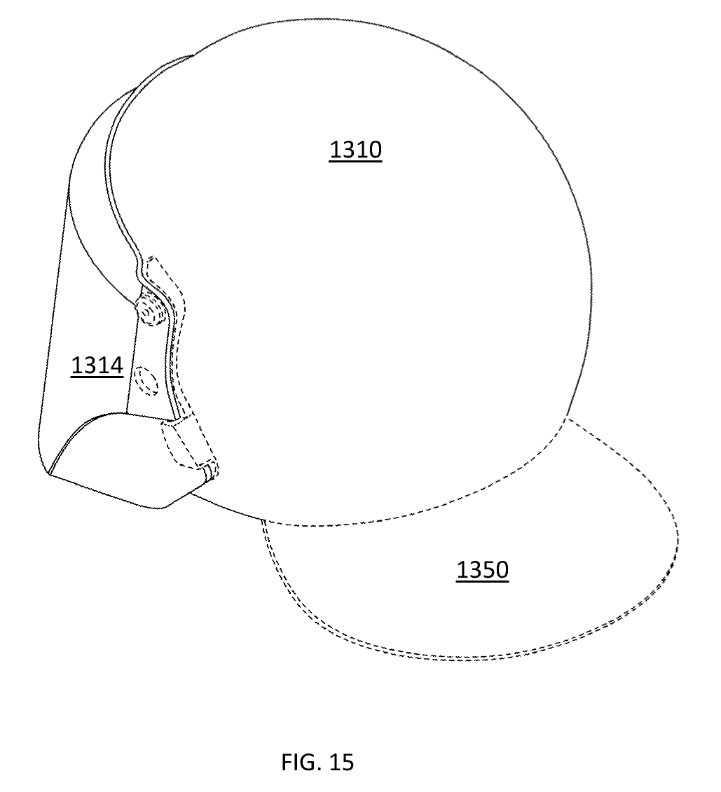

According to yet further exemplary embodiments, as shown in FIG. 13-15 a protection garment may include a head covering 1300, such as a helmet, hat, hood, balaclava or other head covering. The head covering may be or may optionally be used in combination with the hood embodiment described above. In an embodiment used in combination with a hood, such as hood 104, 308 or 804, head covering may optionally be placed over, under, or surrounding the hood. According to some exemplary embodiments, head covering may be a variety of types of helmets, such as a hard-hat helmet, bicycle helmet, motorcycle helmet, welding helmet, ballistic helmet, automobile helmet, flight helmet, sports helmet, or other head covering as would be understood by a person having ordinary skill in the art.

The helmet or head covering 1300 may incorporate conductive material 1310 in accordance with any of the protective garment embodiments described herein. In some embodiments, the conductive material may further include nanotechnology, such as metallic nano-strand conductive composite material or nano-reinforced carbon fiber composite material. However, other embodiments do not utilize nanotechnology in the conductive material. The helmet or head covering may additionally include areas of low or no conductivity 1314. In some embodiments, areas of low or no conductivity may include visors or areas around a user's eyes, ears, nose, and/or mouth in order to prevent damage to sensitive or exposed tissues. The head covering or helmet may additionally include a ground strap 1320 or other conductive connection for guiding a charge to ground or to a grounding strap or conduit disposed on additional protective garments worn by the user, as discussed herein. The head covering or helmet may further include, or be worn in combination with, a heat resistant layer 1340 and/or a water resistant or waterproof layer 1332 described in relation to other embodiments herein, as would be understood by a person having ordinary skill in the art. In some exemplary embodiments, the helmet may further include a protective drape 1350 or covering for a user's neck, which may optionally include electrically conductive material or insulating material.

In another embodiment, a cardiopulmonary protection garment is made of a fabric including electrically conductive metal fibers, such as the moisture-wicking fabrics of the Silverescent® family. The garment is contemplated as having a number of useful forms such as the hooded jacket of FIGS. 1 and 3, the sweater/shirt of FIGS. 11A and 11B, the cape of FIG. 9, the vest of FIG. 10, and the backpack of FIG. 12. The electrically conductive layer may be used alone or combined with additional fabric layers (FIGS. 2 and 4) to improve appearance and/or wearability. In some embodiments, the electrically conductive fabric layer is contoured to avoid the wearer's heart region (see FIG. 3, and FIGS. 6A-6D).

While the invention has been described in relation to the embodiments shown in the accompanying Drawing figures, other embodiments, alternatives and modifications will be apparent to those skilled in the art. It is intended that the Specification be exemplary only, and that the true scope and spirit of the invention be indicated by the following Claims.

1. A protective garment comprising:
a backpack having an electrically conductive material disposed on the backpack for promoting a rapid flashover around the perimeter of the backpack.

2. The protective garment of claim 1, wherein the backpack is configured to direct the flashover away from certain areas by comprising areas of reduced conductivity, wherein no electrically conductive material is disposed.

3. The protective garment of claim 2, wherein the areas of reduced conductivity are adjacent to a user when the backpack is worn to direct the flashover away from the user.

4. The protective garment of claim 3, wherein the areas of reduced conductivity comprise inside surfaces of the shoulder harness, waist strap, or the surface adjacent a wearer's back.

5. The protective garment of claim 1, wherein the electrically conductive material comprises metallic nano-strands.

6. The protective garment of claim 1, wherein the electrically conductive material comprises nano-reinforced carbon fibers.

7. The protective garment of claim 1, further comprising at least one electrically conductive grounding belt.

8. The protective garment of claim 7, wherein the electrically conductive grounding belt is configured to connect to a grounding element directly or through an electrically conductive material disposed on another protective garment worn by a user.

9. The protective garment of claim 1, further comprising a water absorbent outer fabric for supporting a flashover when damp and an inner waterproof lining.

10. The protective garment of claim 1, further comprising electrically conductive material disposed on an outer surface of the waist strap and shoulder harness.

11. A protective garment comprising:
a head covering, wherein an electrically conductive material is disposed on the head covering for promoting a rapid flashover around the perimeter of the head covering.

12. The protective garment of claim 11, wherein the head covering is configured to direct the flashover away from certain areas by comprising areas of reduced conductivity, wherein no electrically conductive material is disposed.

13. The protective garment of claim 12, wherein the areas of reduced conductivity are proximate at least one of a user's eyes, ears, nose, and mouth.

14. The protective garment of claim 11, wherein the electrically conductive material comprises metallic nano-strands.

15. The protective garment of claim 11, wherein the electrically conductive material comprises nano-reinforced carbon fibers.

16. The protective garment of claim 11, further comprising at least one electrically conductive grounding strap.

17. The protective garment of claim 16, wherein the grounding strap is configured to connect to a grounding element directly or through an electrically conductive material disposed on another protective garment worn by a user.

18. The protective garment of claim 11, further comprising a heat resistant lining between the electrically conduct material and a user.

19. The protective garment of claim 11, wherein the protective garment is a helmet.

20. The protective garment of claim 11, wherein the head covering comprises a water absorbent outer layer for supporting a flashover when damp and at least one of a water resistant or waterproof inner layer.
